(19) United States
(12) Patent Application Publication
(10) Pub. No.: US 2019/0033309 A1
(43) Pub. Date: Jan. 31, 2019

(54) METHOD FOR USING BIOLOGICAL MATERIAL FOR DETERMINATION OF DIFFERENCES IN BINDING TO A MOLECULE OF INTEREST

(71) Applicant: Emanuel SMEDS, SÖDRA SANDBY (SE)

(72) Inventors: Emanuel SMEDS, SÖDRA SANDBY (SE); Clément NAUDIN, BROMMA (SE)

(21) Appl. No.: 16/079,577

(22) PCT Filed: Feb. 17, 2017
(86) PCT No.: PCT/SE2017/050152

§ 371 Date: Aug. 24, 2018

(30) Foreign Application Priority Data
Feb. 24, 2016 (SE) 1650243-7

(51) Int. Cl. G01N 33/569; G01N 33/543; G01N 33/566; G01N 33/68

(52) U.S. Cl. CPC G01N 33/569; G01N 33/543; G01N 2500/02; G01N 33/68; G01N 33/566

(57) ABSTRACT

The present invention relates to a method for rapid determination of whether a corresponding ligand from other species or individuals is bound to a molecule that binds to a ligand in one species or individual. The method uses an inhibition assay to make comparisons between different species or individuals within a species and can be used also in a semi-quantitative manner.

### FIELD OF THE INVENTION

The present invention relates to a method for rapid determination if a molecule of interest binds to biological material from various human or animal species or different individuals within a species and the uses thereof.

### TECHNICAL BACKGROUND

Laboratory animals are used in a variety of different types of biomedical research applications. Many animals are used for testing new pharmaceutical compounds for their safety and efficacy. Animals are also used to examine the toxicity of different compounds.

Since much of the biomedical science is focused on finding new treatments for diseases, many experiments with laboratory animals are performed in order to gain more information on how human or animal diseases can be treated or prevented. A number of different laboratory animals are used in these experiments, among the most commonly used are mice, rats, guinea pigs, gerbils, dogs, cats and sheep.

One area where this is evident is in regards to bacterial infectious diseases, where bacterial pathogens or bacterial products such as proteins are administered in laboratory animals and disease progression is monitored. Since bacterial pathogens have a number of molecules to evade the immune system, the interactions between these proteins and proteins in human blood plasma is of particular interest. Bacterial proteins can bind to a variety of different plasma proteins and through various mechanisms help the bacterial pathogen to evade the immune system. To try to develop inhibitors against these bacterial proteins, different strategies have been employed. One approach has been to develop monoclonal or polyclonal antibodies against these proteins.

Another approach has been to develop peptide inhibitors that mimic the plasma target molecule in order to inhibit the binding between the bacterial protein and the plasma protein. Often these experimental inhibitors are then tested in animal models in vivo to try to see if there is a potential as new therapies.

A particular problem is to know if the investigator has selected a suitable laboratory animal species. In a number of scientific studies, it has been established that some bacterial proteins only bind to target ligands from some, but not other species. Here are some examples of these observations in regards to the bacterial pathogen Staphylococcus aureus alone: Staphylococcus aureus staphylococcal superantigen-like protein 7 (SSL7) binds to complement C5 from the following species: human, primate, sheep, pig and rabbit, but not to rat and cow. Staphylococcus aureus Clumping factor A (ClfA) binds to fibrinogen from the following species: human, cow, mouse, dog, cat and pig, but not to sheep. Staphylococcus aureus bone sialoprotein-binding protein (Bbp) binds to fibrinogen from human but not to cat, dog, cow, sheep, mouse or pig fibrinogen.

The observed selectivity is not restricted to S. aureus proteins, it has also been observed with proteins from Streptococcus pyogenes (S. pyogenes) where the protein streptokinase activated plasminogen from some species (human, cat and monkey), but did not activate plasminogen from some other species (mouse, rat, sheep, pig and cow).

The selectivity in the binding of bacterial proteins to plasma proteins from different species creates problems when it comes to testing these bacterial proteins and toxins in laboratory animals. If the bacterial protein is not tested beforehand with the corresponding ligand from the animal's plasma, unnecessary and putatively painful laboratory animal experiments may be a result. One option to circumvent this problem is to initially purify the corresponding protein from plasma from the animal species of interest and study if there is a similar interaction as seen with the other (typically human) protein. It is however, quite laborious and costly to purify a certain protein from a number of different animal species.

Another option is to express recombinant proteins based on the amino acid sequences from different animals, but also this option may be laborious and the resulting proteins may lack the same folding and post-translational modifications that would be found in the native proteins. Recombinant proteins may therefore behave differently as compared to native proteins and hence not perform well in the assays.

There has also been attempts made with assays with monoclonal antibodies generated against a target. However, some targets are difficult or impossible to make a monoclonal antibody against. Further it is costly and laborious to make monoclonal antibodies. In addition, antibodies or antibody fragments may not be able to access a region of interest in a protein due to the large size and structure of both antibodies and antibody fragments.

There is a need for a cost-effective, simple to use method, that does not require extensive laborious work. Also, the method should not be limited to studies of interactions between antibodies from different sources and corresponding antigens.

### SUMMARY OF THE INVENTION

The present invention disclose a method for comparing how biological material from different animal species or different individuals within a species can inhibit the binding between a molecule of interest and a biological molecule, comprising the steps:
A. providing biological material, in a sample earlier obtained, from at least one species and biological material, in a sample earlier obtained, from a specific species;
B. providing at least one dilution of the biological material from the at least one species, and the biological material from the specific species, of step A, further allowing for each of said at least one dilution to be pre-mixed with a molecule M, providing pre-mixed samples comprising molecule M;
C. providing a host ligand L with known affinity for the molecule M of step B, said host ligand L and the pre-mixed samples of step B comprising molecule M, being allowed to interact and thereby final samples are obtained;
D. washing the final samples;
E. detecting a signal from molecule M of the final samples of step D that comprise molecule M bound to the ligand L, preferably molecule M comprise a detectable compound, preferably said compound is selected from the group (detection marker, protein or chemical);
F. comparing said detection signal in an inhibition curve and obtaining a result.

The present invention is performed in vitro, on samples earlier obtained. In one embodiment the at least one dilutions is prepared according to the same ratio for each of the at least one species to be screened as well as for the specific species. In another embodiment the at least one species and the specific species are different individuals of the same species.

In another embodiment the biological material is selected from the group consisting of blood, blood serum, blood plasma, lacrimal fluid, seminal fluid, vaginal fluid, urine and cell lysate from tissues and organs. In one embodiment the biological material is blood. In another embodiment the biological material is blood serum. In another embodiment the biological material is blood plasma. In another embodiment the biological material is cerebrospinal fluid (CSF). In another embodiment the biological material is lacrimal fluid. In another embodiment the biological material is seminal fluid. In another embodiment the biological material is vaginal fluid. In another embodiment the biological material is urine and/or cell lysate from tissues and/or organs.

In another embodiment the molecule of interest is labeled with a luminescent, fluorescent or radioactive compound. In one embodiment the molecule of interest is labeled with a luminescent compound. In another embodiment the molecule of interest is labeled with a fluorescent compound. In another embodiment the molecule of interest is labeled with a radioactive compound.

In another embodiment the molecule of interest is selected from the group consisting of protein, antibody, chemical compound, pharmaceutical compound, toxin, lipid, DNA or RNA molecule, carbohydrate molecule, cell, bacterium, virus, parasite, and fungus. In one embodiment the molecule of interest is a protein. In another embodiment the molecule of interest is an antibody. In another embodiment the molecule of interest is a chemical compound. In another embodiment the molecule of interest is a pharmaceutical compound. In another embodiment the molecule of interest is a toxin. In another embodiment the molecule of interest is a lipid. In another embodiment the molecule of interest is a DNA molecule. In another embodiment the molecule of interest is a RNA molecule. In another embodiment the molecule of interest is a carbohydrate molecule. In another embodiment the molecule of interest is a cell. In another embodiment the molecule of interest is a bacterium. In another embodiment the molecule of interest is a virus. In another embodiment the molecule of interest is a parasite. In another embodiment the molecule of interest is a fungus.

In another embodiment the present invention further comprise a human ligand coupled to solid matter. In another embodiment the human ligand is coupled to agarose beads.

In another embodiment the present invention further comprise the molecule of interest being coated on solid matter. In another embodiment the molecule of interest being coated on plastic. In another embodiment the molecule of interest being coated on agarose beads. In another embodiment the present invention further comprise cycles of animal plasma being applied to the beads. This may imply an attempt to saturate the binding of bacterial protein to animal protein.

In another embodiment the present invention further comprise that antibodies reactive against the molecule of interest has been removed from the biological material.

In another embodiment the results obtained are compared to see which laboratory animal species has antibodies against the molecule of interest. In one embodiment different individual animals within the same species are compared. In one embodiment the biological material from human or animal origin has been fractionated.

In one embodiment the human ligand (pure preparation) is applied and the ratio of free human ligand that goes through the column versus the bound human ligand in the column is compared, since this will indicate if the animal plasma has binding properties.

The present invention disclose a method to test if biological components from a certain species (animal or human) or individuals within a species display similar or different binding to a molecule with binding affinity for a ligand from a certain species. In one embodiment the at least one species is selected from the group consisting of human and animal. In one embodiment the at least one species is selected from the group consisting of human, dog, cat, guinea pig, rat, sheep, horse, monkey, goat, gerbil, chicken, mouse and trout. In one embodiment the specific species is selected from the group consisting of human and animal. In one embodiment the specific species is selected from the group consisting of human, dog, cat, guinea pig, rat, sheep, horse, monkey, goat, gerbil, chicken, mouse and trout.

Another embodiment of the invention is that the ligand protein is bound to some matrix either directly or indirectly. The molecule is then added, followed by biological material that is run over the beads, resulting in that more and more bacterial protein is released from the matrix, either in free form or in complex with the ligand from the biological material being tested. The matrix is then washed from the biological material and remaining molecules bound to the matrix is measured. Said molecule may be measured using methods of detecting luminescence, fluorescence or radioactivity. In one embodiment the molecule of interest is measured using methods of detecting luminescence. In another embodiment the molecule of interest is measured using methods of detecting fluorescence. In another embodiment the molecule of interest is measured using methods of detecting radioactivity. Said molecule may also be detected by antibodies binding to said molecule. Bound antibodies can be detected by using secondary antibodies conjugated to an enzyme. Examples of such enzymes comprise horseradish peroxidase or alkaline phosphatase.

In one embodiment the present invention comprise samples that are in liquid state. This implies that the molecules in the samples are in a soluble state. The sensitivity in the present invention for finding animal plasma samples that are inhibiting the binding of molecule M to a coated ligand L is thereby implied to be high. The sensitivity is implied to be high since the binding of molecule M to animal plasma sample can be performed in solution where neither molecule M nor the animal plasma sample molecules are coated to a surface. Coating of a molecule to a surface may inhibit or even destroy its binding properties towards other molecules and hence performing this step in solution can be advantagous for a number of interactions.

In one embodiment the method is performed using ELISA. In another embodiment the method is performed using affinity chromatography. In another embodiment the method is performed using surface plasmon resonance.

Another aspect of the present invention relates to a screening kit for use according to the method of the present invention, comprising: biological material from at least one species, molecule M and ligand L. One embodiment of the present invention further comprise at least one detection molecule.

Yet another aspect of the present invention relates to use of a method according to the present invention for comparison how biological materials from different animal species or different individuals within a species can inhibit the binding between a molecule of interest and a biological molecule.

Another embodiment of the invention is that biological material with inhibitory activity is used to discover new molecules interfering with the binding site for the described ligand and the uses of these molecules in therapy or diagnosis.

Another embodiment of the invention is to use the method for screening antibodies or sera with inhibitory activities against the interaction of the molecule and ligand of interest.

### DESCRIPTION OF THE DRAWINGS

FIG. 1. Animal plasma inhibition of SSL7 binding to coated human complement C5. Citrated plasma from animals was pre-incubated with SSL7 followed by addition to microtiter plates coated with human complement C5 (hC5) (1 μg/well; 5 pmol). Bound SSL7 was detected by rabbit polyclonal anti-TEV (1:1000) (Pierce) followed by anti-rabbit-HRP conjugate (1:3000) (Bio-Rad). After washing, the absorbance was measured at 450 nm. The assay was performed in triplicate and the results are shown as mean±SD. The relative binding capacity was calculated as described below.

FIG. 2. Animal plasma inhibition of Efb binding to coated human fibrinogen and human C3. (FIG. 2A) Citrated plasma from animals was pre-incubated with Efb followed by addition to microtiter plates coated with human C3 (hC3) (1 μg). (FIG. 2B) Citrated plasma from animals was pre-incubated with Efb followed by addition to microtiter plates coated with human fibrinogen (hFg) (1 μg). (FIG. 2C) Citrated human plasma depleted of either C3 (C3DP) or Fg (FgDP) or normal plasma (Ctrl) was pre-incubated with Efb and added to microtiter plates coated with hFg or hC3. Bound Efb was detected by anti-His HRP conjugate (Abcam) and absorbance measured at 450 nm. The assay was performed in triplicate and the results are shown as mean±SD. The relative binding capacity was calculated as described below.

FIG. 3. Plasma pull-down experiment using agarose beads binding to Efb. Human citrated plasma was added to agarose beads in presence or absence of Efb. Upon washing of the beads, eluted samples were analyzed using 10% SDS-PAGE under reducing conditions (FIG. 3A, FIG. 3B). Samples were also analyzed under reducing conditions using Western blot with an anti-human C3d antibody (1:2000) (DAKO) followed by anti-rabbit-HRP conjugate (1:3000) (Bio-Rad) (FIG. 3C, FIG. 3D). As a control, purified hC3 (1 μg) and hFg (1 μg) was loaded.

FIG. 4. Plasma inhibition assay using lyophilized and frozen citrated plasma. The relative binding capacity was compared using either frozen or resuspended freeze-dried citrated plasma. The different plasma samples were pre-incubated with Efb followed by addition to microtiter plates coated with either human fibrinogen (hFg) (1 μg) or human complement C3 (hC3) (1 μg). Bound Efb was detected by anti-His-HRP conjugate (Abcam) and absorbance measured at 450 nm. The assay was performed in triplicate and the results are shown as mean±SD. The relative binding capacity was calculated as described below.

### DETAILED DESCRIPTION OF THE INVENTION

When a molecule has a determined plasma ligand in a certain animal species, this invention describes a rapid and simple method that can be used to determine if the same ligand in other animal species or in other individuals of the same species is bound or not.

From the results, it is also possible to make semi-quantitative comparisons between the bindings to ligands from different species. The results from the method can be used for several purposes, including identification of suitable laboratory animals to use for testing the molecule. When the method is combined with molecular information on differences between different animal species or different individuals of the same species in regards to a certain protein, it can be used to explain why certain ligands are bound but not others. Molecular information in regards to proteins can be for example differences in amino acid sequences, glycosylation or other post-translational modifications. Molecular information in regards to carbohydrates can for example be structural differences or sulfation patterns. The method can be used to choose suitable laboratory animal species for studying a certain molecule of interest. The method can also be used to identify putative previously unknown binding sites in plasma proteins, by comparison of the plasma proteins from species that bind and not bind respectively to the molecule of interest.

Interactions between molecules and ligands in living organisms form the basis for a number of physiological processes as well as a number of pathological processes. These interactions are typically very specific in the structural requirements for the interactions to occur. Even small differences may prevent the interaction from occurring. In the case of proteins, these small differences may be a result of differences in the amino acid sequence or differences in post-translational modifications such as glycosylations. Typically, any given protein in different animal species will not be identical, there will be some molecular differences and these differences may be the reason why certain interactions may not occur. It is therefore important to be able to know if a molecule can interact with a certain molecule in a given animal species or in an individual within a certain species or not.

In one embodiment the present invention is a method for comparing how biological material from different animal species or different individuals within a species can inhibit the binding between a molecule of interest and a biological molecule, comprising the steps:
A. providing biological material in a sample earlier obtained, from at least one species and biological material in a sample earlier obtained, from a specific species;
B. providing at least one dilution of the biological material from the at least one species, and the biological material from the specific species, of step A, further allowing for each of said at least one dilution to be pre-mixed with a molecule M providing pre-mixed samples comprising molecule M so that the interactions between molecule M and the samples can occur either in solution or on a solid surface, providing pre-mixed samples comprising molecule M;
C. providing a host ligand L with known affinity for the molecule M of step B, said host ligand L and the pre-mixed samples of step B comprising molecule M, being allowed to interact and thereby final samples are obtained;
D. washing the final samples;
E. detecting a signal from molecule M of the final samples of step D that comprise molecule M bound to the ligand L, preferably molecule M comprise a detectable compound, preferably said compound is selected from the group (detection marker, protein or chemical);
F. comparing said detection signal in an inhibition curve and obtaining a result.

One positive aspect of the present invention is that it is very inexpensive to get access to many different proteins, including bacterial proteins, either by recombinant production or by purification. The invention does not require using specific polyclonal or monoclonal antibodies. In addition, antibodies or antibody fragments may not be able to access a region of interest in a protein due to the large size and structure of both antibodies and antibody fragments.

### Methods:

The method has been developed using citrated plasma from different animal species, but is not limited to plasma. The method also applies to other biological materials such as fluids, tissues or cellular sources.

Initially a binding between a molecule (M) and a host ligand (L) has been established by either searching the scientific literature or by performing binding experiments. The host species is often human, but can be any animal species or a transgenic animal. Methods for detection can include for example Western blotting, protein gel electrophoresis, ELISA, surface plasmon resonance or spectroscopy. It can also be one or more individuals within a species.

The molecule may be a biological molecule such as a protein, antibody, chemical compound, pharmaceutical compound, toxin, lipid, DNA or RNA molecule, carbohydrate molecule, cell, bacterium, virus, parasite or fungus.

The method requires some kind of method for detection of the molecule M. Methods for detection can include for example Western blotting, protein gel electrophoresis, ELISA, surface plasmon resonance, spectroscopy and affinity chromatography. Molecule M can be labeled in various manners, including bioluminescence, fluorescence or radioactive labeling. Molecule M may also be detected by binding of other binding partners, including antibodies recognizing either molecule M or a tag that is found on molecule M. Other binding partners may include single chain antibodies, phage display proteins, lectins or other proteins with a specific binding to molecule M.

### Step 1:

Biological materials from different animal species are collected.

In one example, the biological material is citrated plasma from several different animal species.

### Step 2:

The molecule M is pre-mixed with various dilutions of biological material from the different animal species.

As an alternative, instead the concentration of the molecule M can be varied in the different pre-mix experiments.

### Step 3:

The host ligand L is coated or coupled on a surface.

In one example, the surface is a plastic microtiter plate used for ELISA assays and wells in the plate are coated with a purified host ligand: human complement C3 (hC3).

### Step 4:

The pre-mixed samples from step 2 are added to the host ligand L that is already coated on a surface. The sample is incubated for a period of time.

One option is to co-incubate molecule M with animal plasma in one-step and then add the mixture to the plate containing coated ligand. Another option is to perform the co-incubation of molecule M with animal plasma directly on the plate containing the coated ligand.

In one example, a mixture of Efb and various animal plasma samples are added to wells that had been previously coated with human complement C3 (hC3).

### Step 5:

The wells are washed to remove any molecules that have not bound to the coated ligand.

In one example this is done using a buffer at physiological pH.

### Step 6:

Molecules bound to the coated host ligand are detected. Bound molecules can be detected with biochemical methods such as ELISA, Western blotting, surface plasmon resonance, affinity chromatography or spectroscopy.

In one example, bound Efb was detected by anti-His HRP conjugate and absorbance measured at 450 nm in a spectrophotometer. Other methods for detection include surface plasmon resonance, Western blotting, affinity chromatography.

### Step 7:

The results are analyzed and the tested species or tested individuals are grouped into the following three groups.

A) Inhibition is seen when using biological material from a certain species or certain individuals.

An inhibition of the signal from the molecules bound to the ligand in presence of biological material is interpreted as a possibility that this species or individual contain biological ligands that inhibited the binding between the molecule M and the ligand L. The inhibition may be a result of a binding between molecule M and the corresponding ligand from the other species.

B) No inhibition is seen when using biological material from a certain species or certain individual.

If the signal from the molecules bound to the ligand in presence of biological material is not seen, this is interpreted as the possibility that this species or individual does not contain biological ligands that could inhibit the binding between molecule M and the host ligand. If the ligand L is a protein, the lack of inhibition may be a result of molecular differences in amino acid composition or post-translational modifications in the tested species. If ligand L is a polysaccharide, the lack of inhibition may be structural differences, for example in charge and positioning of sulfate groups. In some cases, although more rare, the tested species or individual may lack the corresponding ligand altogether. For example, the method can be used to confirm that a transgenic animal is lacking a certain protein in its citrated plasma or to confirm that a transgenic animal has a certain protein in its citrated plasma.

C) A weak inhibition is seen when using biological material from a certain species or certain individual.

A weak inhibition of the signal from the molecules bound to the ligand in presence of biological material is interpreted as a possibility that this species or individual contain biological ligands that inhibited the binding between the molecule M and the ligand L but the inhibition is not as potent as for the positive control. The weak inhibition may be a result of molecular differences in amino acid composition or post-translational modifications in the tested species that is making the interaction weaker. In some cases, although more rare, the tested species may have a significantly lower concentration of the corresponding ligand.

### Step 8:

Possible uses of the results.

The results from the assay can be used in several different applications. These applications include, but are not limited to these:

A) Identify animal species that are suitable or not suitable for testing of molecule M in vivo. Identify animal species that display an inhibition curve similar to the established original binding species. An inhibition curve can be drawn after performing an experiment with the method described in this application. For example, if citrated plasma from a certain animal species can inhibit the interaction between the molecule M and the ligand L, increasing concentrations of plasma will result in less binding of molecule M to the coated ligand L. The inhibition curve can be transformed into a graph showing relative binding capacity (procedure described below). Only animal species displaying a similar inhibition curve as the original binding species may be expected to present results in further analyses that can be used for predictions in regards to the original binding species. Hence the present invention implies to preferably be using, animal species displaying a similar inhibition curve as the original binding species in animal experiments, when testing the molecule M of interest. These animal species would then also display a similar relative binding capacity since this variable is based upon the inhibition curves. Other animals displaying no or weak inhibition are implied to be avoided in animal experiments with molecule M. This method has the possibility to reduce unnecessary laboratory animal experiments significantly.

B) Prediction of binding site or binding sites in the host ligand L. The information may be used to predict putative binding sites in the host ligand. If the binding site in the host ligand L is not known, molecular comparisons of the amino acid sequences or other differences between animal species plasma that are binding or not binding to the molecule M can be used to predict putative binding sites in the host ligand L.

C) Molecular design of host ligands with increased or reduced binding to molecule M. Identification of species with ligands that bind or not bind to molecule M can be used to design new ligand molecules with desired properties.

If molecular differences can be identified in host ligands with no binding to molecule M, these differences can be used to design a new host ligand with no or very little binding to molecule M.

In one example, this can be used to make point mutations in the amino acid sequence in a host ligand that then looses the binding to molecule M. In another example, this can be used to increase the binding between a host ligand and the molecule M, an application that can be important for both uses in therapy as well as for biotechnological applications.

For example, in a biotechnological application where a protein is purified from a mixture of proteins, an increased binding between the molecule M and the ligand L can be used to increase the yield of the desired protein.

For example, in a medical therapy where a protein binds to a protein ligand found in cancer cells, increased binding can be used to increase the effectiveness of the therapy and lower toxicity in the host given the therapy.

One positive aspect with the present invention is that there is always a specific species that the results from the other species are compared to, so that the specific species will also serve as the positive control that demonstrates that the assay works. If the specific species do not demonstrate an inhibition of binding of molecule M to the coated ligand L, the user will know that there was an error in performing the assay.

### Examples of Applications of the Method

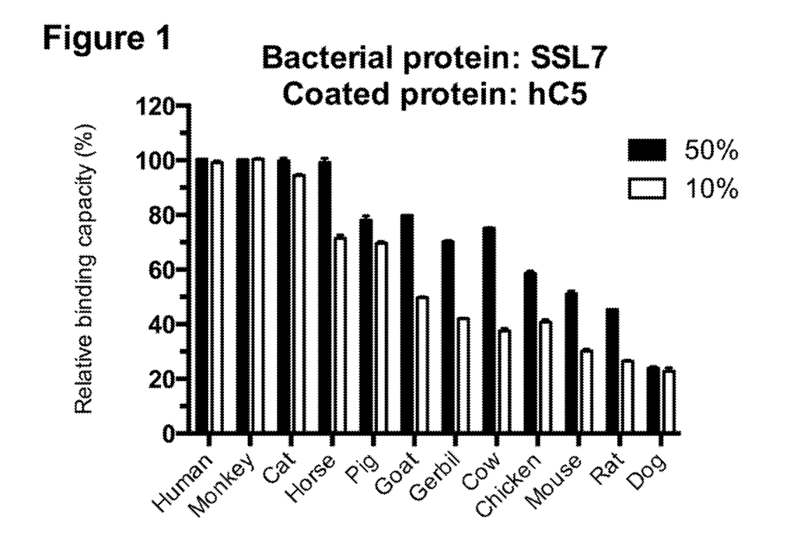

To demonstrate that the method works, the bacterial Staphylococcus aureus protein SSL7 was used. Human complement C5 was coated on microtiter wells. In separate tubes, SSL7 was premixed with various concentrations of citrated plasma from various animal species. From the inhibition experiment, the relative binding capacity for the different samples was calculated as described below. The results are shown in FIG. 1. The obtained results fit very well with the scientific literature, as it has already been demonstrated that SSL7 binds C5 from the following species: human, monkey, pig but not to C5 from rat and cow. As seen in FIG. 1, the relative binding capacity for rat and cow is lower both at 10% and 50% plasma concentration as compared with human, monkey and pig.

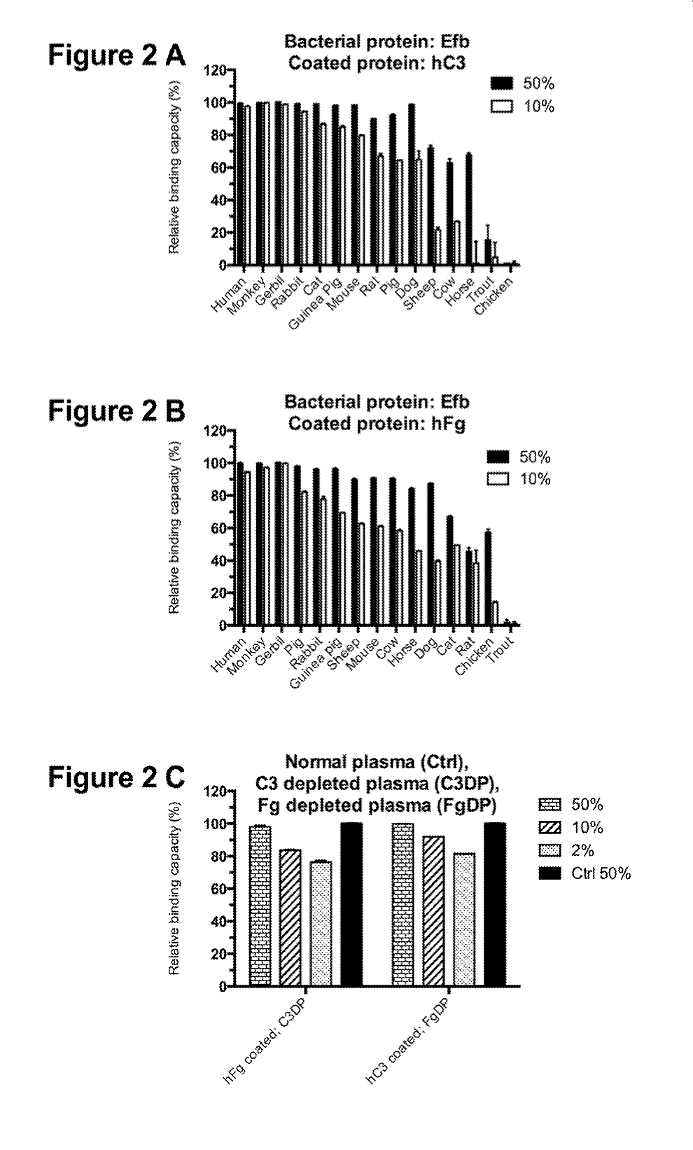

To see that the method was not limited to only one bacterial protein, we also used the method with the Staphylococcus aureus protein Efb. Since the bacterial protein Efb can bind to both human Fg and human C3, it was important to see that the binding results could be valid for both the ligands. Human C3 was coated on microtiter plate wells and in separate tubes, Efb was premixed with different concentrations of citrated plasma from different animal species. The premixed samples were added to the microtiter plates and bound Efb was detected with antibodies (FIG. 2A). Furthermore, human Fg was coated on microtiter plate wells and in separate tubes, Efb was premixed with different concentrations of plasma from different animal species. The premixed samples were added to the microtiter plates and bound Efb was detected with antibodies (FIG. 2B). Interestingly, the patterns with different species in FIGS. 2A and 2B were not identical.

Since the bacterial protein Efb can bind to both Fg and to C3, it was important to see that the binding results were indeed specific for the intended interaction. It was therefore important to demonstrate that the binding of plasma C3 to Efb in the premixing step would not interfere with the binding of Efb to coated human Fg. Similarly, it was therefore important to demonstrate that the binding of plasma Fg to Efb in the premixing step would not interfere with the binding of Efb to coated human C3. To ascertain this, an experiment was performed where human C3 depleted plasma (C3DP) and human Fg depleted plasma (FgDP) was used. In FIG. 2C, human Fg or human C3 was coated onto microtiter plates. Efb was pre-mixed with varying concentrations of human plasma in separate tubes and the mixtures were then added to the wells. Upon washing, bound protein was detected using antibodies. As expected, C3-deficient plasma still inhibited the binding of Efb to coated human Fg and Fg-deficient plasma still inhibited the binding of Efb to coated human C3 (FIG. 2C).

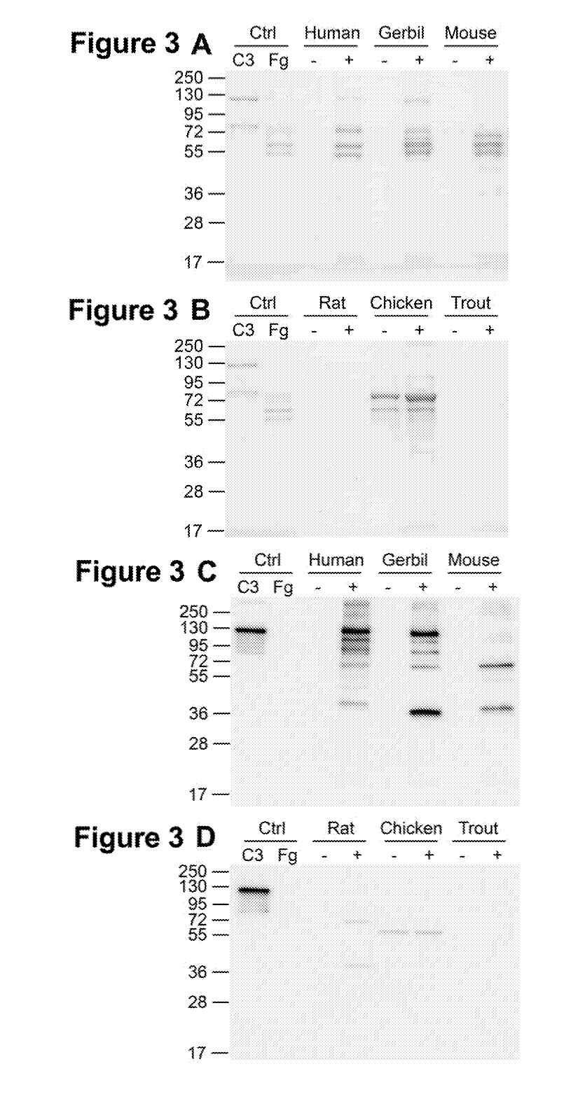

To further confirm that the assay works, still another method was used to confirm the results. For this assay, agarose beads coupled to anti-His antibodies were used. The beads were incubated with Efb containing His-tag followed by incubation with human plasma. Beads were then washed and bound proteins eluted from the beads. Eluted proteins were analyzed using SDS-PAGE under reducing conditions and some animal samples eluted proteins that are consistent in size with human Fg and human C3. The binding to these plasma proteins appeared to be specific for Efb, since the control beads without Efb did not elute these proteins (FIG. 3A, FIG. 3B).

In the case of chicken, the sample clotted and the results should therefore be interpreted with caution. To further corroborate the findings using SDS-PAGE, a Western Blot analysis was performed on the eluted material from the beads (FIG. 3C, FIG. 3D). Samples were run under reducing conditions on SDS-PAGE followed by transfer of proteins to a membrane. The results corroborated that the novel method is working as intended, since trout plasma had no relative binding capacity to hC3 or hFg. Rat plasma had poor binding capacity to both hFg and hC3, but Western Blot indicated possible presence of C3 fragments. The results corroborated that the novel method is working as intended.

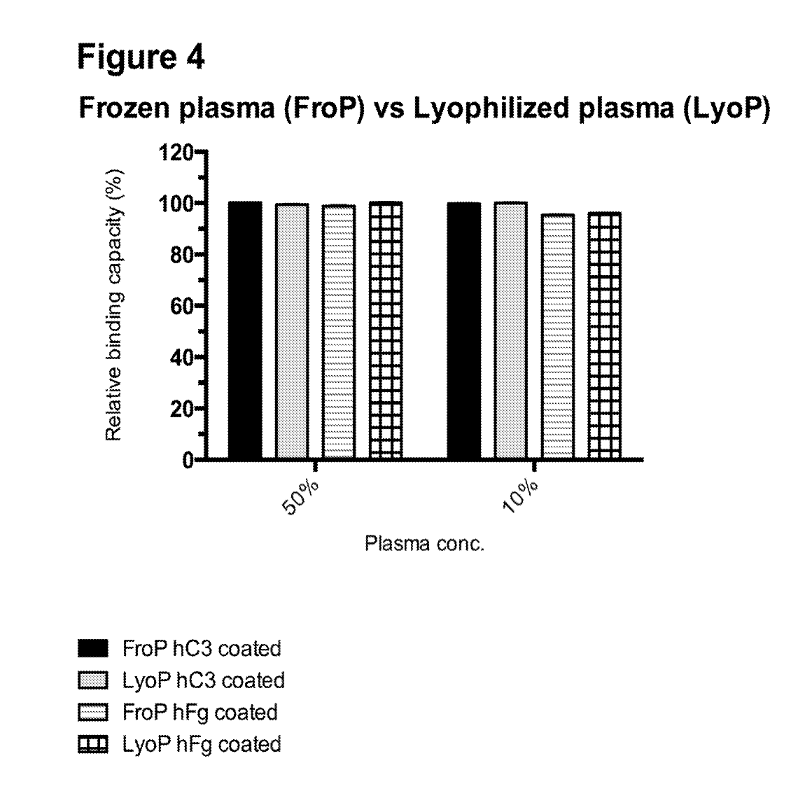

As it would be significantly more cost-effective to ship plasma samples in a freeze-dried form, this was also analyzed (FIG. 4). Microtiter plates were coated with either hC3 or hFg. In the assay either normal citrated frozen plasma was thawed and used (FroP in the graph) or plasma was lyophilized and resuspended in liquid (LyoP in the graph) and pre-mixed with Efb. As can be seen in the graph, lyophilized plasma had retained the relative binding capacity both in the case of Fg and C3 interactions with Efb.

### Calculation of Relative Binding Capacity

Since the assay is based upon an inhibition assay where a reduction in signal actually indicates a binding of the bacterial protein to one or more plasma ligands from the animal plasma being tested. Hence, the inhibition is converted into a term called relative binding capacity. Complete binding of bacterial protein to plasma from a tested animal would yield 100% relative binding capacity.

A=absorbance with only plasma present, but no bacterial protein. Microtiter plate coated with the purified plasma ligand.
B=absorbance without plasma, but in presence of bacterial protein. Microtiter plate coated with the purified plasma ligand.
C=absorbance without plasma, but in presence of bacterial protein. Microtiter plate has no coated ligand.
D=absorbance upon premixing of the bacterial protein with plasma from a certain species at a desired plasma concentration (i.e. mouse plasma 50%).

Microtiter plate has coated ligand. Typically several different plasma concentrations from different animal species are used in the assay.

Upon premixing of the bacterial protein with plasma from a certain species (i.e. mouse plasma 50%) an absorbance value D is read in the spectrophotometer.
The relative binding capacity E in percent is then defined as:

E=100−(D−A)/(B−C)*100

E can then be defined for different species and different concentrations of plasma, for example 50% mouse plasma. Note: in our assays shown in the figures, the value C was so low (always even lower than A) that it was disregarded in the calculations.
There are many positive aspects of the present invention, it is performed with simultaneous mixing of molecule M with animal samples rather than with two or more steps. The present invention further implies an increased sensitivity since molecule M binds to animal proteins in solution, as compared to interactions with coated protein. The present invention does not require use of monoclonal or polyclonal antibodies or fragments thereof. Further, the present invention discloses to work well with purified proteins that are coated as ligands on a plate. And the present invention has a built in positive control using samples from the specific species and then comparing the specific species to different animal species.

1. Method for comparing how biological material from different animal species or different individuals within a species can inhibit the binding between a molecule of interest and a biological molecule, comprising the steps:
A. providing biological material in a sample earlier obtained, from at least one species and biological material in a sample earlier obtained, from a specific species;
B. providing at least one dilution of the biological material from the at least one species, and the biological material from the specific species, of step A, further allowing for each of said at least one dilution to be pre-mixed with a molecule M, providing pre-mixed samples comprising molecule M;
C. providing a host ligand L with known affinity for the molecule M of step B, said host ligand L and the pre-mixed samples of step B comprising molecule M, being allowed to interact and thereby final samples are obtained;
D. washing the final samples;
E. detecting a signal from molecule M of the final samples of step D that comprise molecule M bound to the ligand L, preferably molecule M comprise a detectable compound, preferably said compound is selected from the group (detection marker, protein or chemical);
F. comparing said detection signal in an inhibition curve and obtaining a result.

2. Method according to claim 1, wherein the at least one dilutions is prepared according to the same ratio for each of the at least one species to be screened as well as for the specific species.

3. Method according to claim 1, wherein the biological material is selected from the group consisting of blood, blood serum, blood plasma, cerebrospinal fluid (CSF), lacrimal fluid, seminal fluid, vaginal fluid, urine and cell lysate from tissues and organs.

4. Method according to claim 1 wherein the molecule of interest is labeled with a luminescent, fluorescent or radioactive compound.

5. Method according to claim 1 wherein the molecule of interest is selected from the group consisting of protein, antibody, chemical compound, pharmaceutical compound, toxin, lipid, DNA or RNA molecule, carbohydrate molecule, cell, bacterium, virus, parasite, and fungus.

6. Method according to claim 1, further comprising a human ligand coupled to solid matter preferably agarose beads.

7. Method according to claim 1, further comprising the molecule of interest being coated on solid matter, preferably plastic, preferably on agarose beads, further comprising cycles of animal plasma being applied to the beads.

8. Method according to claim 1, further comprising that antibodies reactive against the molecule of interest has been removed from the biological material.

9. Method according to claim 1, wherein the results obtained are compared to see which animal species have antibodies against the molecule of interest, preferably different individual animals within the same species are compared.

10. Screening kit for use according to the method of claim 1, comprising:
biological material from at least one species, molecule M and ligand L, preferably further comprising at least one detection molecule.

11. Use of a method according to claim 1 for comparison how biological materials from different animal species or different individuals within a species can inhibit the binding between a molecule of interest and a biological molecule.
